(19) United States
(12) Patent Application Publication
(10) Pub. No.: US 2004/0249147 A1
(43) Pub. Date: Dec. 9, 2004

(54) Synthesis of key azole-antifungal intermediates

(76) Inventors: Jitendra Sattigeri, Haryana (IN); Jasbir Singh Arora, Delhi (IN); Sanjay Malhotra, Delhi (IN); Ashwani Kumar Verma, Delhi (IN); Mohammad Salman, Plainsboro, NJ (US)

Correspondence Address: RANBAXY INC., 600 COLLEGE ROAD EAST, SUITE 2100, PRINCETON, NJ, 08540, US

(21) Appl. No.: 10/490,540

(22) PCT Filed: Sep. 24, 2002
(86) PCT No.: PCT/IB02/03942

(30) Foreign Application Priority Data
Sep. 25, 2001 (IN) 982/DEL/2001

(51) Int. Cl. C07D 471/02; C07D 413/02; C 07D 4 5/02

(52) U.S. Cl. 544/147; 544/284; 546/82; 544/310; 544/374; 548/257; 548/517; 548/311.1

(57) ABSTRACT

Methods for producing azole compounds useful as intermediates for antifungal compounds and compositions including reaction of epoxy alcohols with reactants having active hydrogen attached to nitrogen, oxygen or sulfur in the presence of redox coupling agents. In some embodiments, a procedure known as a Mitsunobu reaction is utilized for the transformations.

### CROSS-REFERENCE TO RELATED APPLICATIONS

This application claims priority under 35 U.S.C. §119 from Indian patent application number 982/DEL/2001, filed on Sep. 25, 2001.

### FIELD OF THE INVENTION

The invention relates to methods of production of azole compounds useful as antifungal therapeutic agents. More particularly, the invention relates to methods of making intermediates which are useful for producing antifungal azole compounds.

### BACKGROUND OF THE INVENTION

Compounds of Formula I have been found to be important intermediates for the syntheses of azole antifungal compounds, for example those of Formula II.

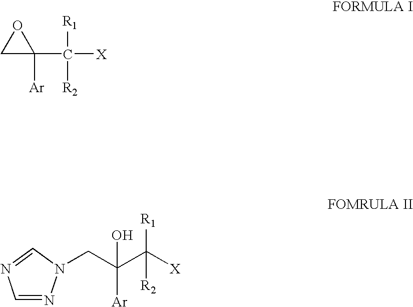

A process for the preparation of a particular antifungal compound of Formula I, shown as Formula III in Scheme I, wherein R₁=CH₃, R₂=H, X=

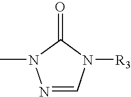

has been reported in U.S. Pat. Nos. 5,371,101 and 6,034,248 to Itoh et al. assigned to Takeda Chemical Industries, Ltd. (Itoh et al. '101 and Itoh et al. '248, respectively) This intermediate has been used to prepare several compounds, including those known as TAK-187 and TAK-456 of Formulae IV and V, respectively. These are potent antifingal compounds.

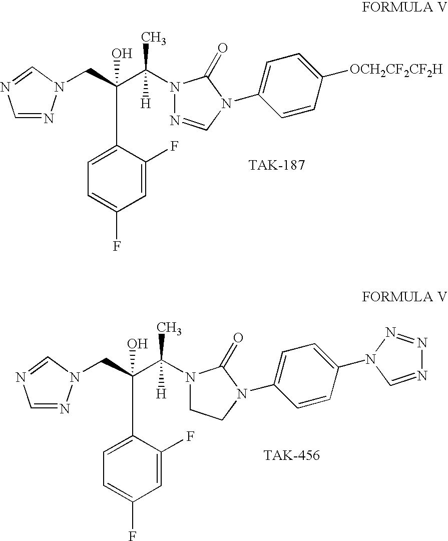

The synthetic route described in Itoh et al. '101 is a multistep process in which an epoxy alcohol of Formula VII, (Route 1 in Scheme 1) of required stereochemistry (1S,2R) is activated as a triflate (Formula VII) with trifluoromethane sulphonic acid anhydride and subjected to nucleophilic substitution with various nucleophiles, including triazolone derivatives. The labile nature of triflates can result in less than optimal yields for such processes.

Also, these processes require specialized equipment to carry out commercial scale reactions at temperatures as low as −70 to −80° C. Consequently, Itoh et al. presents an alternate route (Route 2), requiring conversion of (1R,2R) epoxy alcohol of Formula VI to another epoxy intermediate of Formula IX. This modification introduced two more steps in this process and also requires the use of an expensive compound, R-ethyl lactate, as starting material.

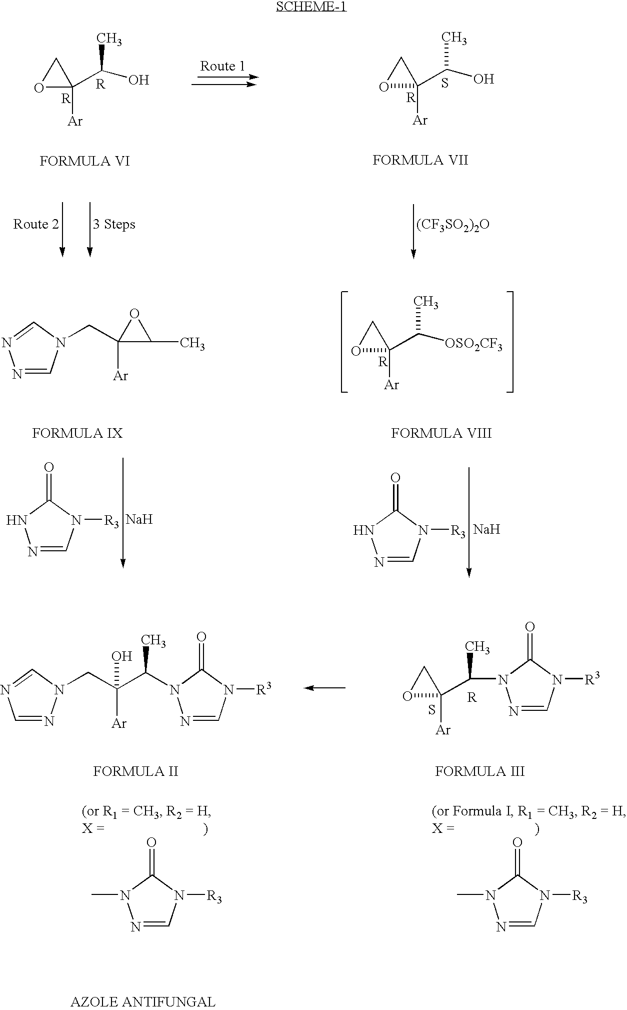

### SUMMARY OF THE INVENTION

The present invention provides a simple, one-pot, single-step, efficient and commercially viable process for the preparation of compounds of Formula I. The process is improved and commercially advantageous over known processes. The process according to the description provided herein avoids disadvantages associated with prior art process. These disadvantages include the necessity of using very low temperature equipment, low yields, and an excessive number of process steps.

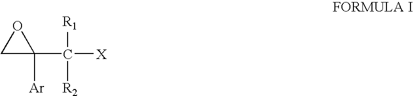

Compounds of Formula I can serve as key intermediates for the synthesis of azole antifungal compounds of Formula II. It is thus desirable to provide a process for the preparation of compounds of Formula I.

A particular embodiment of the transformation from a compound of Formula X to a compound of Formula I is exemplified in Scheme 2.

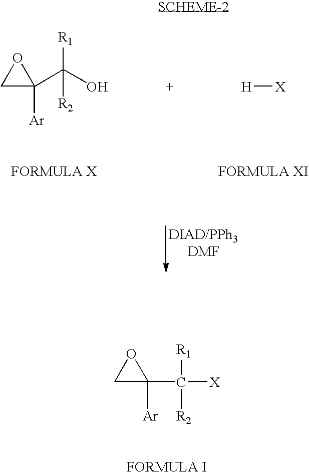

In the above structures, the substituent Ar is an aromatic hydrocarbon group (for example, phenyl) having one to three substituents independently selected from halogen (for example, fluorine, chlorine, bromine or iodine), halogenated lower (C₁₋₃) straight or branched alkyl, and halogenated lower (C₁₋₃) straight or branched alkoxy group. Particular examples of Ar include: 2,4-difluorophenyl, 2,4-dichlorophenyl, 4-chlorophenyl, 4-fluorophenyl, 2-chlorophenyl, 4-trifluoromethylphenyl, 2-fluoro-4-chloro-phenyl, 3-chloro-4-fluorophenyl, 4-trifluoromethoxyphenyl, 2,4,6-triflurophenyl, and 4-bromophenyl. In some embodiments, preferred Ar include a phenyl group with one to two halogen atoms, and preferred halogens are fluorine and chlorine. In some embodiments, 2,4-difluorophenyl is particularly preferred.

In the above structures, the substituents R₁ and R₂ are independently selected from the group consisting of hydrogen, straight or branched alkyl groups having 1 to 3 carbon atoms, for example, methyl, ethyl, propyl or isopropyl. In some embodiments, preferred alkyls for R₁ and R₂ are methyl and ethyl. Preferred combinations of R₁ and R₂ are hydrogen and hydrogen; hydrogen and methyl; methyl and methyl. In some particularly preferred embodiments, R ₁ is methyl and R₂ is hydrogen.

In the above structures, the substituent X can be —O—R, —S—R (wherein R is optionally substituted aliphatic, or optionally substituted aromatic hydrocarbon, as defined below), or a nitrogen-containing substituent, where nitrogen bonds to either H in Formula XI or carbon in Formula I, such as cyclic amide, imide, urea, triazolones including

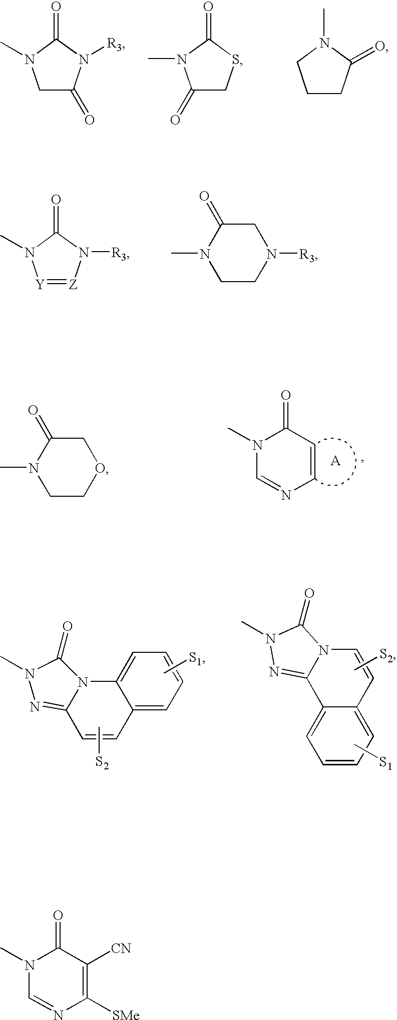

The substituent R₃ is a group bonded through a carbon atom such as optionally substituted aliphatic or aromatic hydrocarbon residues (as defined for R below) and optionally substituted aromatic heterocyclic groups. The optionally substituted aromatic heterocyclic groups include optionally substituted fused or non-fused aromatic heterocyclic groups having at least one hetero atom selected from nitrogen, sulphur and oxygen. Examples of the heterocyclic groups include imidazolyl, triazolyl, tetrazolyl, pyrazolyl, pyridyl, thiazolyl, thiadiazolyl, thienyl, furyl, pyrrolyl, pyrazinyl, pyrimidinyl, oxazolyl, isoxazolyl, benzimidazolyl, imidazopyrimidinyl, imidazopyridinyl, imidazopyrazinyl, imidazopyridazinyl, benzothiazolyl, quinolyl, isoquinolyl, quinazolinyl or indolyl. In some preferred embodiments, optionally substituted five or six membered aromatic hetrocyclic groups having 1 to 3 hetero atoms selected from a nitrogen atom, sulphur atom and oxygen atom, such as imidazolyl, triazolyl, thiazolyl, thiadiazolyl, thienyl, furyl, pyridyl or pyrmidinyl can be used. Examples of the substilients for the optionally substituted aliphatic or aromatic hydrocarbon residues and the optionally substituted aromatic heterocyclic groups shown by R₃ can include those defined immediately below for R.

As set forth above, substituent R can be an optionally substituted aliphatic or aromatic hydrocarbon residues. Examples of the optionally substituted aliphatic hydrocarbon residues which can constitute substituent R include alkyl (for example, straight or branched alkyl groups having 1 to 12 carbon atoms such as methyl, ethyl, propyl, butyl, pentyl, hexyl, heptyl, octyl, nonyl, decyl or dodecyl), with lower alkyl groups having 1 to 4 carbon atoms (e.g. methyl, ethyl, propyl or butyl) being present in some preferred embodiments; cycloalkyl (for example, cycloallcyl groups having 3-8 carbon atoms such as cyclopropyl, cyclobutyl, cyclopentyl, cyclohexyl, cycloheptyl or cyclooctyl), with cycloalkyl groups having 3 to 6 carbon atoms (e.g. cyclopropyl, cyclobutyl, cyclopentyl or cyclohexyl) being present in some preferred embodiments; alkenyl (for example, alkenyl groups having 3 to 4 carbon atoms such as propenyl and butenyl), with alkenyl groups having 3 carbon atoms (e.g. propenyl) being present in some preferred embodiments); alkynyl (for example, alkynyl groups having 3 to 4 carbon atoms such as propynyl or butynyl), with alkynyl groups having 3 carbon atoms (e.g. propynyl) being present in some preferred embodiments. Any of the above aliphatic hydrocarbon residues may be substituted, as detailed below.

Examples of the optionally substituted aromatic hydrocarbon residues which can constitute substituent R include optionally substituted aryl groups having 6 to 14 carbon atoms, for example, phenyl, naphthyl, biphenyl, anthryl, or indenyl. In some preferred embodiments, aryl groups having 6 to 10 carbon atoms (e.g. phenyl or naphthyl) are present. Any of the above aromatic hydrocarbon residues may be substituted, as detailed below.

Examples of the substituents for the optionally substituted aliphatic or aromatic hydrocarbon residues include: hydroxy group; optionally esterified carboxy group (for example, methoxycarbonyl, ethoxycarbonyl, propoxycarbonyl or butoxy carbonyl); nitro group; amino group; acylamino group (for example, alkanoyl amino group including acetylamino, propionylamino or butyrylaino); alkylamino group (for example, methylamino, dimethylamino, diethylamino or dibutylamino); optionally substituted cyclic amino group (for example, pyrrolidinyl, morpholino, piperidino, piperazinyl, N-benzylpiperazinyl, N-acetyl piperazinyl, N-aryl piperazinlyl, N-(p-alkoxyphenyl); piperazinyl (for example, N-(p-methoxyphenyl)piperazinyl); N-(p-haloalkoxyphenyl)piperazinyl (for example, N-[p-(2,2,3,3-tetrafluoropropoxy)phenyl]piperazinyl); N-(p-halophenyl)piperazinyl (for example, N-(p-chlorophenyl)piperazinyl); N-(p-alkylphenyl)piperazinyl (for example, N-(p-methylphenyl)piperazinyl); pyrazolinyl or perhydroazepinyl; alkoxy group (for example, methoxy, ethoxy, propoxy or butoxy); aralkyloxy; halogen (for example, fluorine, chlormie or bromine); alkyl; haloalkyl group (for example, trifluoromethyl, dichloromethyl or trifluoroethyl); haloalkoxy group (for example, trifluoromethoxy, 1,1,2,2-tetrafluoroethoxy, 2,2,2-trifluoroethoxy, 2,2,3,3-tetrafluoropropoxy 2,2,3,3,3-pentafluoropropoxy, 2,2,3,3,4,4,5,5-octafluoropentoxy or 2-fluoroethoxy); oxo group; thioxo group; mercapto group; alkyl thio group (for example, methylthio, ethylthio, or butylthio); alkyl sulphonyl group (for example, metlianesulphonyl, ethanesulphonyl or butane sulphonyl); and alkanoyl group (for example, acetyl, formyl, propionyl or butyryl).

Included among the groups which can constitute the substituent R are the following general structures:

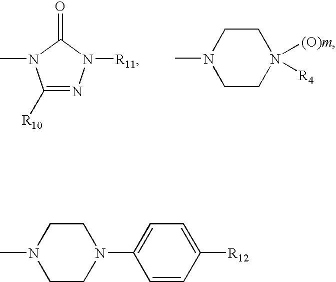

where R₁₀ represents hydrogen or methyl; R₁₁ represents hydrogen, isopropyl, cyclopentyl, 3-hydroxy-2-butyl, or 2-hydroxy-2-butyl, or 2-hydroxy-3-pentyl; m represents 0 or 1; and R₁₂ represents halogen, C₁-C₄ haloalkyl, C₁-C₄ haloalkoxy, nitro, amino, cyano, or a group of formula

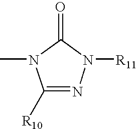

The substituents can also include any of the optionally substituted fused or non-fused aromatic heterocyclic group as defined herein for R₃ or the groups defined herein for R₁₂

In some preferred embodiments, X is

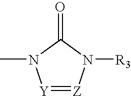

The substituents Y and Z are independently a nitrogen atom or a methine group or a methylene group which may optionally be substituted with a lower alkyl group.

More preferably X

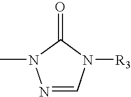

in the compound of Formula I which can be represented by the compound of Formula III as depicted below

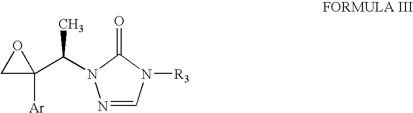

(or Formula I, wherein R₁=CH₃, R₂=H,X;

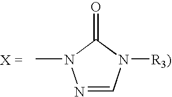

wherein Ar and R₃ are as defined above.

The substituents S₁ and S₂ are independently selected from the group consisting of hydrogen, C₁-C₄ alkyl, C₁-C₄ halo alkyl, C₁-C₄ alkoxy, C₁-C₄ halo alkoxy, halogen, nitro or cyano, open chain amides, optionally substituted aromatic heterocyclic group including optionally substituted fused or non-fused aromatic heterocyclic groups having at least one heteroatom selected from a nitrogen atom, sulphur atom and oxygen atom. These groups can include those as described herein for R₃.

The substituent A represents a benzene ring or a 5- or 6-membered heterocyclic ring wherein one or more of the ring atoms are selected from the group consisting of N, O and S (as described herein for R₃), and the said rings can be optionally fused to a benzene ring or to a 5- or 6-membered heterocyclic ring containing one or more heteroatoms selected from N, O and S, and A can be unsubstituted or have 1, 2, 3 or 4 substituents W in any of the rings.

The substituent W represents C₁-C₄ alkyl, C₃-C₆ cycloalkyl, C₁-C₄ haloalkyl, C₁-C₄ alkoxy, C₁-C₄ haloalkoxy, halogen, nitro, cyano, hydroxy, benzyloxy, hydroxymethyl, a group —NR₄R₅, a group —CONR₄R₅, a group —CH₂—OCO—R₄, a group —CO—R₄, a group —COO—R₄, a group —SO₂R₆, a group —C (═NR₄) NHR₇, a group —C(═NR₇)OR₄, and additionally W can also represent 1-pyrrolyl, 1-imidazolyl, 1H-1,2,4-triazol-1-yl, 5-tetrazolyl (optionally substituted with C₁-C₄ alkyl), 1-pyrrolidinyl, 4-morpholinyl, 4-morpholinyl-N-oxide, or a group —B—R₈.

The substituent R₄ represents hydrogen, C₁-C₄ alkyl, C₃-C₆ cycloalkyl or aryl, wherein aryl represents phenyl or phenyl substituted with one or more C₁-C₄ alkyl, halogen, C₁-C₄ haloalkyl, C₁-C₄ alkoxy or C₁-C₄ haloalkoxy groups.

The substituent R₈ represents hydrogen, C₁-C₄ alkyl, C₃-C₆ cycloalkyl, a group —COR₄ or a group —COCF₃; R₆ represents C₁-C₄ alkyl; R₇ represents hydrogen, —CONH₂, —COMe, —CN, —SO₂NHR₄, —SO₂R₄, —OR₄, —OCOR₄ or —(C₁₋₄ alkyl) —NH.

The substituent B represents a single bond, —O—, —SO₂, —NR₄— or —(C═O)—.

The substituent R₈ represents an aralkyl or a phenyl group optionally substituted with one or more groups R₉.

The substituent R₉ represents C₁-C₄ alkyl, C₃-C₆ cycloalkyl, C₁-C₄ haloalkyl, C₁-C₄ alkoxy, C₁-C₄ haloalkoxy, halogen, nitro, cyano, a group —NR₄R₅, a group —CONR₄R₅, a group —COO—R₄, a group —SO₂R₆, a group —C (═NHR₄) NHR₇, a group —C (═NR₇) OR₄, a phenyl group (optionally substituted with a group C₁-C₄ alkyl, C₁-C₄ haloalkyl, C₁-C₄ alkoxy, C₁-C₄ haloalkloxy, halogen, nitro or cyano).

The substituent R₁₀ represents hydrogen or methyl.

The substituent R₁₁ represents hydrogen, isopropyl, cyclopentyl, 3-hydroxy-2-butyl, or 2-hydroxy-2-butyl, or 2-hydroxy-3-pentyl.

The label m represents an integer 0 or 1.

The substituent R₁₂ represents halogen, C₁-C₄ haloalkyl, C₁-C₄ haloalkoxy, nitro, amino, cyano, or a group of formula

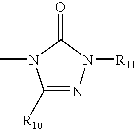

Formula I has two asymmetric centers and thus there are four possible stereoisomers, that is, (1R,2R), (1R,2S), (1S,2R) and (1S,2S). This invention relates to the process for the manufacture of mixture of stereoisomers as well as individual stereoisomers and the most preferred stereoisomer is (1R,2S).

As used herein, a substituent containing an “active hydrogen” refers to a substituent which contains a reactive hydrogen atom. Examples include, but are not limited to, substituents of structure HO—, HS—, and HN—, and can include substituents “X” as described herein.

Unless otherwise defined, all technical and scientific terms used herein have the same ordinary meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

According to embodiments described herein, a specific reaction protocol has been utilized to replace the hydroxyl group of epoxy alcohols with a nucleophilic group in the presence of redox coupling agents with complete inversion of stereochemistry in one simple step. In some embodiments the reaction protocol is known as the Mitsunobu reaction protocol, using Mitsunobu reaction conditions. One of ordinary skill in the art will recognize suitable reagents and conditions for the transformation of alcohols to the desired products. According to some embodiments described herein, the alcohol is activated in situ, followed by the attack of an in situ generated anion of a nucleophile. Nucleophilic attack of compounds of Formula XI (for example, 1 ,2,4-triazole compounds) on particular epoxide alcohols of Formula X affords desired antifungal intermediates in high enantiopurity.

More particularly, the present invention provides a process for the preparation of the compound of Formula I, as shown in Scheme 2, wherein R₁, R₂, Ar and X are as defined earlier, comprising reacting epoxy alcohol of Formula X (which can be prepared, for example, according to procedures disclosed in U.S. Pat. No. 6,133,485 to Singh et al.), with a reactant H-X of Formula XI having active hydrogen attached to nitrogen, oxygen or sulphur atoms, wherein X is as defined earlier, in a suitable solvent in the presence of redox couple agent. The redox couple agent may be any of those known in the art as suitable for this type of coupling.

In some preferred embodiments, the reducing agent is a phosphine. The phosphine is selected from a trialkylphosphine, a triaryl phosphine, or any phosphine with a combination of alkyl and aryl substituents, wherein the aryl may be optionally substituted phenyl or heteroaryl having 1 to 3 heteroatoms selected from the group of N, O and S or a polymer bound phosphine, for example, polymer bound triphenylphosphine.

In some preferred embodiments, the oxidizing agent is selected from the group consisting of dialkylazodicarboxylate, a diallcylazodicarboxamide, N,N,N′,N′-tetrasubstituted azodicarboxamide (for example, N,N,N′,N′-tetramethyl azodicarboxamide (TMAD) and 4,7-Dimethyl-3,5,7-hexahydro-1,2,4,7-tetrazocin-3,8-dione (DHTD)) or a polymer bound methyl azodicarboxylate [such as described in J. Am. Chem. Soc., pp 3973-3976 (1989)]. In some particularly preferred embodiments, the oxidizing agent is diethylazodicarboxylate (DEAD) or diisopropylazodicarboxylate (DIAD). Those of ordinary skill in the art will recognize other reducing and oxidizing reagents which can be adapted to this reaction, including those mentioned in Herr, Albany Molecular Research Technical Reports, 3 (19), 1-36 (1999). This is not to limit the scope of the invention, and any other oxidizing agent can be used to those skilled in the art.

The solvent may be selected from ethers, including diethylether, diisopropylether, tert-butylniethyl ether, or tetrahydrofuran (THF) and the like, from chlorinated solvents including dichloromethane, chloroform, dichloroethane, and the like, from polar aprotic solvents including N,N-dinietlhyl formamide (IMF), N-methyl pyrrolidone, dimethylsulphoxide (DMSO), and the like, or from hydrocarbons including toluene and the like. Alternatively, a mixture of such solvents may be used.

The reaction is carried out at a selected temperature ranging from 0° C. to 100° C., preferably at 0-40° C. and more preferably, at 0-30° C. during a period of one to several hours. More preferably, the reaction is carried out in N,N-dimethyl formamide or tetrahydrofuran in the presence of diisopropyl or diethyl azodicarboxylate and triphenyl phosphine. The desired compound of Formula I is isolated by conventional methods including extraction with at least one suitable solvent selected from the group consisting of dichloromethane, dichloroethane, chloroform, ether, isopropylether, toluene, methyl acetate, ethyl acetate and butyl acetate.

Using the general method described above, epoxy derivatives of Formulae XII and XIII

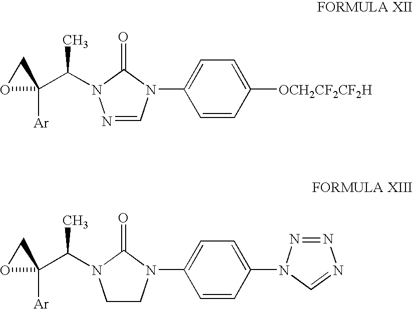

can be synthesized in efficient manner. These are important intermediates for the manufacture of TAK-187 and TAK-456. Similarly SCH-42427 of Formula XIV

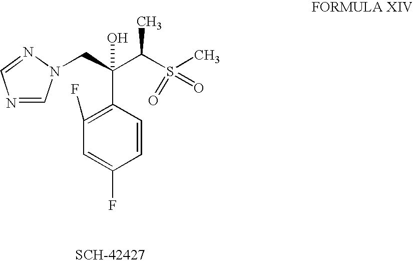

can be prepared using an appropriate thiol derivative (where HX is CH₃SH), followed by oxidation.

The generality of this reaction leads to the application of a wide variety of active hydrogen compounds of the following exemplary structures that can be coupled to epoxy alcohol.

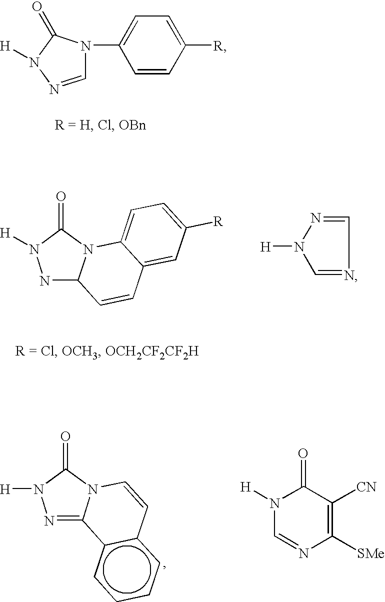

The above mentioned approach makes it possible to synthesize various other azole antifungals such as UR 9746, UR 9751, and Sankyo's compound of formulae XV, XVI and XVII, respectively.

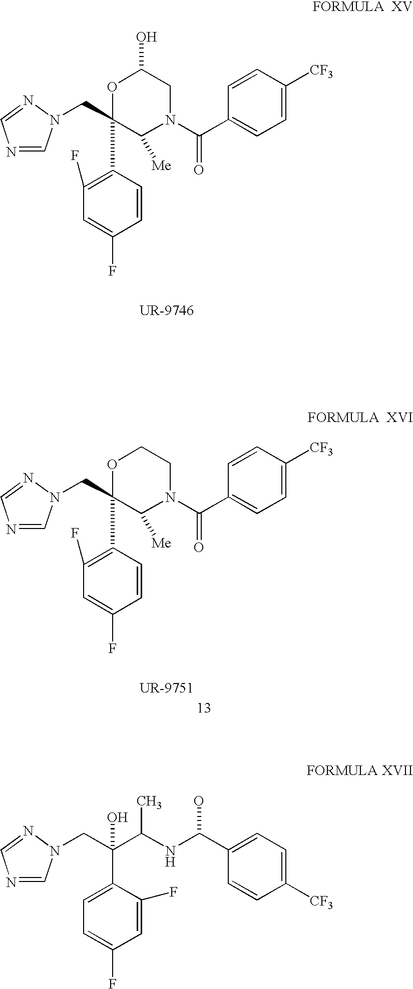

The scope of this invention also covers the process for the synthesis of azole antifungals, but not limited, to those described in U.S. Pat. Nos. 5,545,652 to Itoh et al.; U.S. Pat. No. 5,495,024 to Itoh et al.; U.S. Pat. No. 5,177,094 to Itoh et al., U.S. Pat. No. 6,184,396 to Takeda et al., U.S. Pat. No. 5,888,941 to Bartroli et al., and International Patent Application WO 97/05130.

The invention will be further described in the following examples, which do not limit the scope of the invention described in the claims.

### EXAMPLES

### Example 1

### General Procedure

In a 3 neck 50 ml round bottom flask equipped with guard tube, septum and N₂ inlet, was placed nucleophile (X-H) (1.1 mmol), triphenylphosphine (1.2 mmol) in dry DMF (3-5 ml). The suspension/solution so obtained was then cooled to 0° C. and to this was then added a solution of epoxy alcohol of Formula I (1.0 mmol) in DMF (1 ml) at 0° C. The epoxy alcohols were generally prepared as described in U.S. Pat. No. 5,371,101 to Itoh et al. To this suspension/solution was added diisopropyl azodicarboxylate (DIAD, 1.2 mmol) and the reaction mixture so obtained was stirred at 25-30° C. for about 5-10 hours. After the reaction was over, the reaction mixture was poured into ice-cooled water and extracted with ethyl acetate. The combined ethyl acetate layer was washed with brine and solvent was recovered from the organic phase under vacuum. The residue so obtained was purified by column chromatography (silica gel, 100-200 mesh, 25% ethylacetate/hexane) to afford the desired title compound. Product purities were >95% as determined by ¹H NMR.

The following examples present the ¹H NMR spectral data and product yields of several exemplary compounds prepared according to the procedure in Example 1:

### Example 2

### Preparation of 2-[(1R,2S)-2-(2,4-difluorophenyl-2,3-epoxy-1-methylpropyl]-4-[4-(4-chlorophenyl)-1-piperazinyl]phenyl]-3(2H, 4H)-1,2,4-triazolone (Compound No.1)

¹H NMR (CDCl₃): 1.46 (3H, d, J=7.0 Hz), 2.87, (1H, d, J=4.6 Hz), 3.15 (1H, d, J=4.6 Hz), 3.29-3.35 (8H, m), 4.95 (1H, q, J=7.0 Hz), 6.80-7.68 (12H, m); product yield 34%.

### Example 3

### Preparation of 1-[(1R,2R)-2-(2,4-difluorophenyl)-2,3-epoxy-1-methylpropyl]-6-thiomethyl-5-cyayopyrmidin-4-one (Compound No.2)

¹H NMR (CDCl₃): 1.38 (3H, d, J=6.3 Hz), 2.61 (3H, s), 2.97 (1H, brs), 3.22 (1H, brs) 5.44 (1H, q, J=6.4 Hz), 6.82-(1H, m), 6.96 (1H, m), 7.63 (1H, m), 8.58 (1H,S); product yield 75%.

### Example 4

### Preparation of 2-[(1R, 2R)-2-(2,4-difluorophenyl)-2,3-epoxy-1-methylpropyl]-3-oxo-6-chloro-8-methyl-2,3-dihydro-1,2,4-triazolo[4,3-a]quinoline (Compound No.3)

¹H NMR(CDCl₃): 1.44 (3H, d, J=7.05 Hz), 2.46 (3H, s), 2.87 (1H, d, J=4.65), 3.13 (1H, d, J=4.6 Hz), 5.10 (1H, q, J=7.0 Hz)), 6.76-6.87 (3H, m), 7.29-7.38 (1H, m), 7.51-7.65 (1H, m), 7.66 (1H, s), 9.00 (1H, d, J=8.9 Hz); product yield 41%.

### Example 5

2-[(1R,2R)-2-(2,4-difluorophenyl)-2,3-epoxy-1-methylpropyl]-3-oxo-6-methoxy-8-methyl-2,3-dihydro-1,2,4-triazolo[4,3-a]quinoline (Compound No.4)

¹HNMR(CDCl₃): 1.50 (3H, d, J=7.1 Hz), 2.46 (3H, s), 2.86 (1H, d, J=4.7 Hz), 3.13 (1H, d, J=4.65 Hz), 3.90 (3H, s), 5.12 (1H, q, J=7.1 Hz), 6.76-6.85 (3H, m), 7.12-7.16 (2H, m), 7.30-7.35 (1H, m), 8.98 (1H, d, J=9.36 Hz); product yield 52%.

### Example 6

### Preparation of 2-[(1R, 2R)-2-(2,4-difluorophenyl)-2,3-epoxy-1-methylipropyl]-3-oxo-2,3-dihydro-5-triazole[4,3-a]isoquinoline (Compound No.5)

¹HNMR(CDCl₃): 1.54 (3H, d, J=7.0 Hz), 2.91 (1H, d, J=4.7 Hz), 3.22 (1H, d, J=4.7 Hz), 5.09 (1H, q, J=7.0 Hz), 6.76-6.81 (3H, m), 7.27-7.33 (1H, m), 7.49-7.63 (4H, m), 8.25 (1H, d, J=7.2 Hz); product yield 34%.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A process for the preparation of a compound of Formula I, 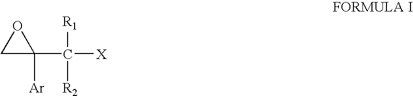
comprising reacting an epoxy alcohol of Formula X 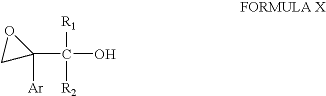
with a reactant of Formula XI
H—X FORMULA XI
in the presence of redox coupling agent comprising a reducing agent and an oxidizing agent for a time sufficient to form a compound of Formula I, wherein
Ar is an aromatic hydrocarbon group having one to three substituents independently selected from halogen, halogenated lower (C₁₋₃) alkyl, halogenated lower (C₁₋₃) alkoxy group;
R₁ and R₂ are independently selected from the group consisting of hydrogen, straight or branched alkyl groups having 1 to 3 carbon atoms; and
X is selected from the group consisting of —O—R, —S—R, and the nitrogen-containing substituents cyclic amide, imide, urea, triazolones, and the following structures: 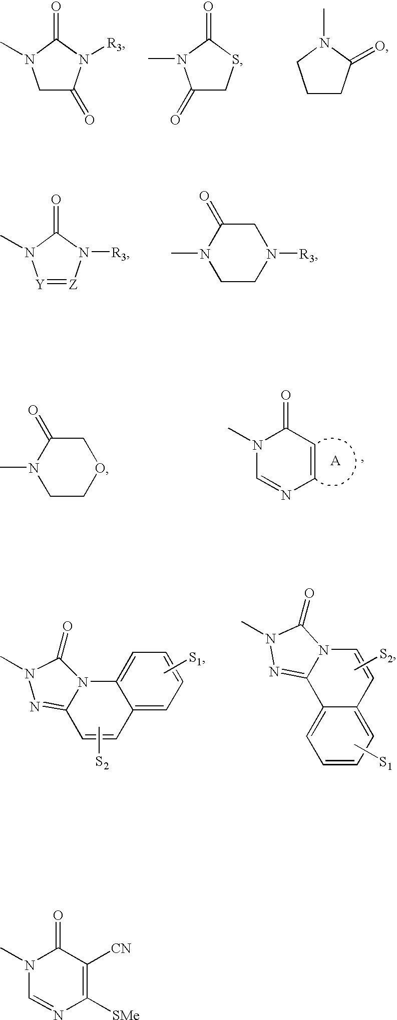
wherein
R is an optionally substituted aliphatic or optionally substituted aromatic hydrocarbon;
R₃ is selected from the group consisting of optionally substituted aliphatic hydrocarbon residues, optionally substituted aromatic hydrocarbon residues and optionally substituted aromatic heterocyclic residues, bonded through a carbon atom;
Y and Z are independently a nitrogen atom, a methine group, or a methylene group optionally substituted with a lower alkyl group;
S₁ and S₂ are independently selected from the group consisting of hydrogen, C₁-C₄ alkyl, C₁-C₄ halo alkyl, C₁-C₄ alkoxy, C₁-C₄ halo alkoxy, halogen, nitro or cyano, open chain amides, optionally substituted aromatic heterocyclic group including optionally substituted fused or non-fused aromatic heterocyclic groups having at least one heteroatom selected from a nitrogen atom, sulphur atom and oxygen atom;
A is a benzene ring or a 5- or 6-membered heterocyclic ring wherein one or more of the ring atoms are selected from the group consisting of N, O and S, and the ring can be optionally fused to a benzene ring or to a 5- or 6-membered heterocyclic ring containing one or more heteroatoms selected from N, O and S, and wherein A can be unsubstituted or have 1, 2, 3 or 4 substituents W in any of the rings,
wherein W is selected from the group consisting of C₁-C₄ alkyl, C₃-C₆ cycloalkyl, C₁-C₄ haloalkyl, C₁-C₄ alkoxy, C₁-C₄ haloalkoxy, halogen, nitro, cyano, hydroxy, benzyloxy, hydroxymethyl, a group —N R₄R₅, a group —CONR₄R₅, a group —CH₂—OCO—R₄, a group —CO—R₄, a group —COO—R₄, a group —SO₂R₆, a group —C (═NR₄) NHR₇, a group —C(═NR₇)OR₄, 1-pyrrolyl, 1-imidazolyl, 1H-1,2,4-triazol-1-yl, 5-tetrazolyl (optionally substituted with C₁-C₄ alkyl), 1-pyrrolidinyl, 4-morpholinyl, 4-morpholinyl-N-oxide, and a group —B—R₈;
R₄ is selected from the group consisting of hydrogen, C₁-C₄ alkyl, C₃-C₆ cycloalkyl, phenyl, or phenyl substituted with one or more C₁-C₄ allyl, halogen, C₁-C₄ haloalkyl, C₁-C₄ alkoxy or C₁-C₄ haloalkoxy groups;
R₅ is selected from the group consisting of hydrogen, C₁-C₄ alkyl, C₃-C₆ cycloalkyl, a group —COR₄ or a group —COCF₃;
R₆ is C₁-C₄ alkyl;
R₇ is selected from the group consisting of hydrogen, —CONH₂, —COMe, —CN, —SO₂NHR₄, —SO₂R₄, —OR₄, —OCOR₄ and —(C₁₋₄ alkyl)—NH;
B is selected from the group consisting of a single bond, —O—, —SO₂, —NR₄— or C═O)—;
R₈ is selected from the group consisting of an aralkyl or a phenyl group optionally substituted with one or more groups R₉; and
R₉ is selected from the group consisting of C₁-C₄ alkyl, C₃-C₆ cycloalkyl, C₁-C₄ haloalkyl, C₁-C₄ alkoxy, C₁-C₄ haloalkoxy, halogen, nitro, cyano, a group —NR₄R₅, a group —CONR₄R₅, a group —COO—R₄, a group —SO₂R₆, a group —C(═NHR₄) NHR₇, a group —C (═NR₇) OR₄, and a phenyl group optionally substituted with a group C₁-C₄ alkyl, C₁-C₄ haloalkyl, C₁-C₄ alkoxy, C₁-C₄ haloalkloxy, halogen, nitro or cyano.

2. The process of claim 1, wherein X is selected from the cyclic amide, imide, urea, triazolones, and the following structures:

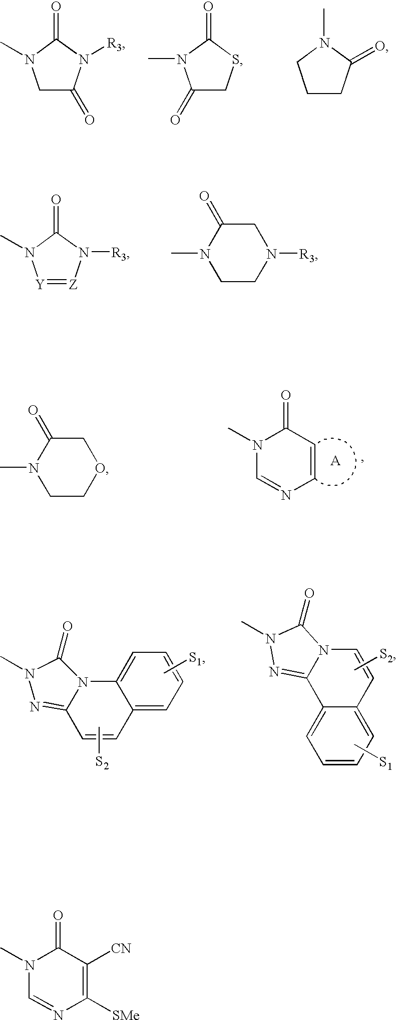

3. The process of claim 2, wherein X is 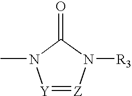
wherein Y is nitrogen atom and Z is methine.

4. The process of claim 1, wherein Ar is 2,4-difluorophenyl.

5. The process of claim 1, wherein R₁ is methyl and R₂ is hydrogen.

6. The process of claim 3, wherein R₃ is a substituted or unsubstituted cyclic amino group.

7. The process of claim 6, wherein the amino group is selected from pyrrolidinyl, morpholino, piperidino, piperazinyl, N-benzylpiperazinyl, N-acetyl piperazinyl, N-aryl piperazinyl, N-(p-alkoxyphenyl)piperazinyl, N-(p-haloalkoxyphenyl)piperazinyl N-(p-halophenyl)piperazinyl, N-(p-alkylphenyl)piperazinyl, pyrazolinyl, perhydroazepinyl, haloalkyl, haloalkoxy, each of which can be substituted or unsubstituted.

8. The process of claim 3, wherein R₃ is a substituted aryl group.

9. The process of claim 8, wherein R₃ is an aryl group substituted with a group selected from the group consisting of haloalkoxyl and tetrazolyl.

10. The process of claim 1, wherein the reducing agent is a phosphine is selected from trialkylphosphine, triaryl phosphine, wherein the aryl may be optionally substituted phenyl or heteroaryl having 1 to 3 heteroatoms selected from the group of N, O and S or a polymer-bound phosphine comprising polymer bound triphenylphosphine.

11. The process of claim 1, wherein the oxidizing agent is selected from the group consisting of dialkylazodicarboxylate, a dialkylazodicarboxamide or a polymer-bound methyl azodicarboxylate.

12. The process of claim 1, wherein the reaction of a compound of Formula X and a compound of Formula XI is carried out in a solvent is selected from ethers, chlorinated solvents, polar aprotic solvents and hydrocarbon solvents.

13. The process of claim 12, wherein the solvent is selected from at least one of the group consisting of diethylether, diisopropylether, t-butylmethyl ether, tetrahydrofuran, dichloromethane, chloroform, dichloroethane, N,N-dimethyl formamide, N-methylpyrrolidone, dimethyl sulphoxide and toluene.

14. The process of claim 1, wherein the reaction is carried out in N,N-dimethylformamide or tetrahydrofuran in the presence of diisopropyl- or diethyl azodicarboxylate and triphenyl phosphene.

15. The process claim 1, wherein the reaction is carried out at a temperature ranging from 0° C. to 100° C.

16. The process of claim 15, wherein the reaction is carried out at a temperature 0-40° C.

17. A compound of Formula I prepared by the process of claim 1.

18. The compound of claim 17, wherein X is 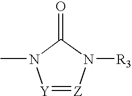
wherein R₃ is selected from the group consisting of optionally substituted aliphatic or aromatic hydrocarbon residues and optionally substituted aromatic heterocyclic group, bonded through a carbon atom; and
wherein Y and Z are independently a nitrogen atom or a methine group or a methylene group optionally substituted with a lower alkyl group.
